(19) United States
(12) Patent Application Publication
(10) Pub. No.: US 2007/0219439 A1
(43) Pub. Date: Sep. 20, 2007

(54) Spectral Photometry Method for Determining the Oxygen Saturatiobn of the Blood in Optically Accessible Blood Vessels

(75) Inventors: Walthard Vilser, Rudolstadt (DE); Martin Hammer, Jena (DE)

Correspondence Address: REED SMITH, LLP;ATTN: PATENT RECORDS DEPARTMENT, 599 LEXINGTON AVENUE, 29TH FLOOR, NEW YORK, NY, 10022-7650, US

(73) Assignee: IMEDOS GmbH

(21) Appl. No.: 10/594,871

(22) PCT Filed: Mar. 31, 2005
(86) PCT No.: PCT/DE05/00588

§ 371 Date: Sep. 28, 2006

(30) Foreign Application Priority Data
Mar. 31, 2004 (DE) 10 2004 016 435.5

(51) Int. Cl. A61B 5/00

(52) U.S. Cl. 600/323

(57) ABSTRACT

The invention relates to a spectral photometry method for determining the oxygen saturation of the blood in optically accessible blood vessels, by determining the intensity of the reflection of the blood vessels and of their environment that is devoid of vessels, using at least two spectrally diverse images. The aim of the invention is to reduce the stress on the patient during the capture of the spectrally diverse images, achieving at the same time an improved signal-to-noise ratio. In addition, the improved method aims to guarantee a clear association of arteries and veins in the images and to deliver more meaningful values for the oxygen saturation. To capture the spectrally diverse images, the blood vessels and their environment are simultaneously illuminated by illumination radiation of at least one measuring wavelength and at least one reference wavelength, each measuring and reference wavelength being tuned to a respective color channel of a color camera that captures the images, in order to be received by said color channel.

### CROSS-REFERENCE TO RELATED APPLICATIONS

This application claims priority of International Application No. PCT/DE2005/000588, filed Mar. 31, 2005 and German Application No. 10 2004 016 435.5, filed Mar. 31, 2004, the complete disclosures of which are hereby incorporated by reference.

### BACKGROUND OF THE INVENTION

a) Field of the Invention

The invention is directed to a method for the spectral photometric determination of the oxygen saturation of the blood in optically accessible blood vessels by determining the intensity of the reflection of the blood vessels and their vessel-free environment based on at least two spectrally different images and on an empirically determined relationship between the oxygen saturation and a ratio of the intensities of the reflection of the blood vessels and their vessel-free environment. The method according to the invention is provided in particular for application to the fundus of the human eye but is not limited thereto.

b) Description of the Related Art

The oxygen saturation of a hemoglobin sample can be determined in principle by comparing the spectrum of a sample to the spectra of completely oxygenated and completely reduced hemoglobin because, as is generally known, the absorption spectrum of the red blood pigment hemoglobin changes with oxygen saturation.

For example, in Appl. Opt. 27, 1988, 1113-1125, Delori describes a method, based on the Lambert-Beer law, for oximetry in retinal vessels using measurements in three wavelengths to compensate for dispersion losses.

A large number of other methods and devices for oximetry in the ocular fundus which are based on the Lambert-Beer law and are known, e.g., from DE 199 20 157 A1, U.S. Pat. No. 4,253,744, U.S. Pat. No. 4,305,398, U.S. Pat. No. 4,485,820, U.S. Pat. No. 5,119,814, U.S. Pat. No. 5,308,919, U.S. Pat. No. 5,318,022, U.S. Pat. No. 5,776,060, and U.S. Pat. No. 5,935,076 have the disadvantage that the very complex process of light propagation in a blood vessel embedded in the retina and in the environment of this blood vessel is modeled only insufficiently. Consequently, inaccurate and sometimes false values result for oxygen saturation.

DE 102 17 543 A1 describes a method which makes it possible to determine oxygen saturation by comparing a measured spectrum with the spectra of oxygenated and reduced hemoglobin in four wavelengths. Disturbances such as the absorption of other pigments and dispersion in the tissue are compensated through a linear transformation of the logarithmized spectra.

It is disadvantageous that the four wavelengths lie in a spectral region which is highly absorbent for blood. Because of the low signal-to-noise ratio caused by this, it is difficult to achieve the required high accuracy in the reflection measurements at vessels of the fundus.

For determining oxygen saturation in a method disclosed in WO 00/06017 A1, an intermediate image taken of the fundus by a fundus camera is divided into two images which are filtered in such a way that the two images have different wavelengths which are optimized for the electronic recording with respect to the oxygen saturation of the blood. The images are evaluated so as to determine the reflection of the blood vessel and that of its environment. Finally, the oxygen saturation values are determined on the basis of empirical relationships between oxygen saturation and an optical density ratio resulting from the contrast between the blood vessel and its environment.

A disadvantage of this method consists in that a quantitative measurement of oxygen saturation is possible only in veins for which the optical density ratio of an associated artery is known with inhalation by the patient of pure oxygen. The disadvantageous results are as follows:
every patient must inhale oxygen for the examination,
the person conducting the examination must classify the blood vessels as veins or arteries, but
a definitive correspondence of arteries and veins in the images is possible only with additional expenditure.
   Moreover, the method is not completely independent from the melanin pigmentation of the fundus.

### OBJECT AND SUMMARY OF THE INVENTION

On this basis, it is the primary object of the invention to improve the method mentioned above in such a way that the stress on the patient while recording the spectrally different images is reduced and an improved signal-to-noise ratio is achieved at the same time. Further, the improved method should provide more meaningful values for the oxygen saturation and facilitate association of arteries and veins in the images.

In the above-mentioned method for spectral photometric determination of the oxygen saturation of the blood in optically accessible blood vessels, the above-stated object is met in that the blood vessels and their environment are illuminated simultaneously by at least one measurement wavelength and at least one reference wavelength of an illumination beam for recording the spectrally different images, and in that every measurement wavelength and reference wavelength is tuned, respectively, to a color channel of a color camera used to record the images in order to be received by this color channel.

The measurement wavelength is preferably a wavelength at which the reflection of oxygenated and reduced hemoglobin differs, and the reference wavelength is an isosbestic wavelength of the hemoglobin.

It is particularly advantageous that the stress on the patient due to the illumination is substantially reduced by limiting the illumination beam on the illumination side to the selected spectral portions of the illumination beam which are correlated to the color channels of the color camera. Further, this step has advantageous results for the attainable signal-to-noise ratio.

The oxygen saturation is determined as a linear function of the quotient of the logarithmized reflection ratios in the vessel-free environment and on the blood vessel at the measurement wavelength and at the isosbestic wavelength. The slope and linear term of the linear function are determined empirically from readings at a plurality of blood vessels.

The correctives that are empirically determined and taken into consideration additively as means to compensate for disturbances caused by a dependency of the oxygen saturation on the vessel diameter and on the pigmentation of the environment of the blood vessels are particularly advantageous.

The two correctives are linear functions of the respective disturbance—vessel diameter and pigmentation—to be compensated. The slope and linear term of the two linear functions are determined empirically. The pigmentation of the environment of the blood vessels is determined by the logarithm of the quotient of the reflection values of the environment of the blood vessels at the measurement wavelength and at the isosbestic wavelength.

The method according to the invention is preferably so conceived that arteries and veins are distinguished based on the quotient of the logarithmized reflection ratios in the vessel-free environment of the blood vessel and on the blood vessel at the measurement wavelength and at the isosbestic wavelength.

The blood vessels, their direction and their vessel-free environment can be detected automatically by image-processing means or manually. In this way, specular reflections on the blood vessels can be identified and eliminated.

In an advantageous manner when measuring the reflection values perpendicular to the direction of the blood vessel, an average is taken over the reflection values of all of the image points associated with the blood vessel. A plurality of reflection values which are averaged perpendicular to the direction of the blood vessel can be determined along the direction of the blood vessel and the average is taken over these averaged reflection values.

In a special development of the invention, the oxygen saturation is determined in reaction to physiological provocation or stimulation. This can be carried out in different ways, e.g., by flicker light, by inhalation of oxygen or carbogen by the test subject.

A method which is particularly suitable for optical influence consists in that light from at least one light source is modified through programming techniques by a light manipulator arranged in an illumination beam path of an image-generating device, and the modified light is used for illumination and for selective provocation or stimulation.

The oxygen saturation determined by the method according to the invention can be used in a variety of ways for diagnostic purposes.

The invention is further directed to a method of the type mentioned in the beginning for the spectral photometric determination of the oxygen saturation of the blood in optically accessible blood vessels in which the oxygen saturation is determined as a linear function of the quotient of the logarithmized reflection ratios in the vessel-free environment and on the blood vessel at a measurement wavelength at which the reflection of oxygenated and reduced hemoglobin differs and at an isosbestic wavelength of the hemoglobin as reference wavelength, and the slope and the linear term of the linear function are determined empirically from readings at a plurality of blood vessels.

Disturbances due to a dependency of the oxygen saturation on the vessel diameter and on the pigmentation of the environment of the blood vessels can be compensated by empirically determined correctives which are to be taken into account additively.

The invention will be described more fully in the following with reference to the schematic drawings.

### BRIEF DESCRIPTION OF THE DRAWINGS

FIG. 1 shows a simplified view of the construction of an image-generating device for implementing the method according to the invention;

FIG. 2 shows the position of selected wavelength ranges in the color channels when the wavelength ranges prepared on the illumination side are adapted to the color channels with respect to color matching; and

FIG. 3 shows the spatial distribution of the reflection of an artery and a vein in a biological object at a measurement wavelength and a reference wavelength as a section perpendicular to the blood vessels and the averages of the reflections on the blood vessels and in their environment.

### DESCRIPTION OF THE PREFERRED EMBODIMENTS

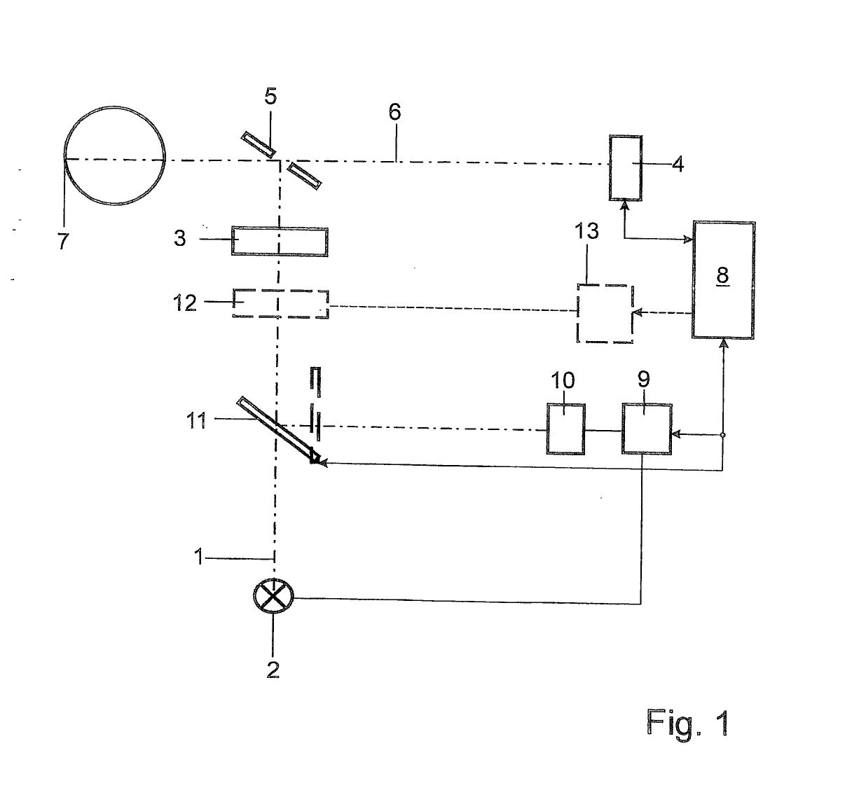

The image-generating device shown in a simplified view in FIG. 1 can be used to implement the method according to the invention which can be applied preferably, but not exclusively, to blood vessels of the ocular fundus.

In principle, the method according to the invention can be applied to optically accessible (and identifiable) blood vessels of biological objects of which the congruent monochromatic images, preferably in different spectra, which are required for the spectral photometric determination of the oxygen saturation of the blood can be recorded, for example, also with a slit lamp, an endoscope or a surgical microscope.

According to the present embodiment example, the images of the fundus of the eye are recorded at a measurement wavelength λₘ=610 nm at which the absorption/reflection of oxygenated and reduced hemoglobin differs and at a reference wavelength, i.e., an isosbestic wavelength λᵢ=548 mm of the hemoglobin.

This may be carried out, for example, with a simple retina camera, shown in FIG. 1, which has been modified in an extremely economical manner and whose illumination system contains in a common illumination beam path 1 at least one illumination source 2 and, particularly for implementing the method according to the invention, a filter device 3 which prepares wavelengths on the illumination side which are spectrally tuned to the color channels of an electronic color camera 4. Further, one of the components known from retina camera technology is a perforated mirror 5. A recording beam path 6 passes through the central opening of this perforated mirror 5. The illumination light is directed through optically imaging elements, not shown here, to the fundus 7 and particularly to the blood vessels located therein and their environment over an area surrounding the central opening. Light reflected by the fundus 7 passes along the recording beam path 6 and along optically imaging elements, not shown, to an image-generating recording system. In the present embodiment example, the color camera 4 is provided for this purpose. The camera control of the color camera 4 is connected to a central controlling and evaluating unit, particularly a controlling and evaluating computer 8. A power supply 9 serving to supply power to the two illumination sources 2 and 10 is also connected to the controlling and evaluating computer 8 and likewise corresponding tilting mirror controls.

It is not important as regards the invention whether only the continuous illumination source 2 is used or only the illumination source 10 which is constructed as a strobe illumination source is used, or whether both sources 2 and 10 are used together as is shown in FIG. 1. The means for coupling the latter into the common illumination beam path 1, which is carried out conventionally in this instance by a swing-out mirror 11, is also not important as regards the invention.

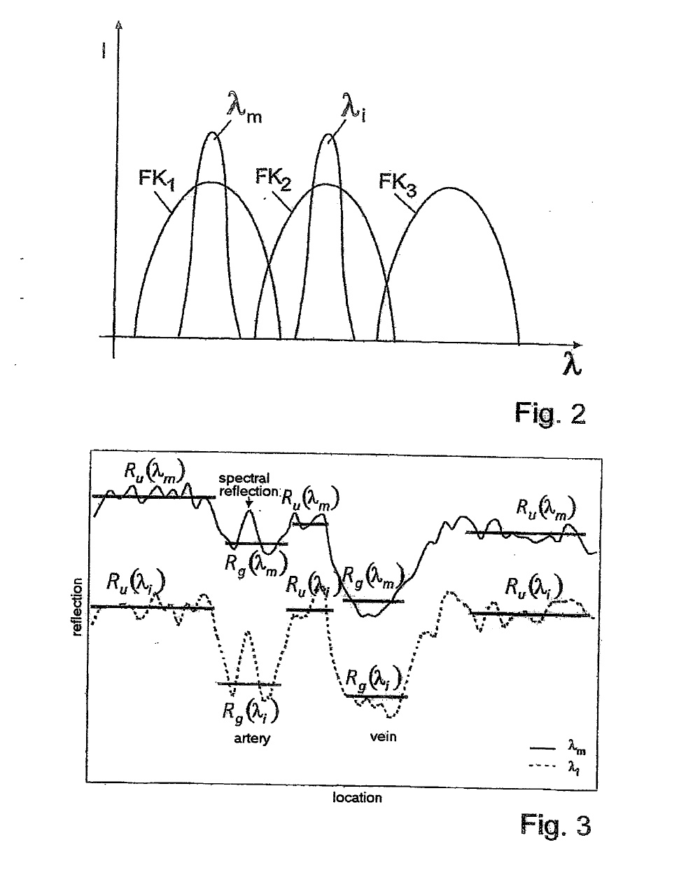

However, it is important that the filter device 3 is selected based on the spectral characteristic of the color camera 4 and is inserted in the illumination beam path 1 so that at least the measurement wavelength λₘ and the reference wavelength λᵢ can be generated for simultaneous illumination of the fundus 7 in diverse colors, each of these wavelengths being tuned to one of the color channels FKⱼ (j=1, 2, 3) of the color camera 4 with respect to a color matching corresponding to FIG. 2.

Suitable optical filters 3 are layer filters such as dual bandpass filters or triple bandpass filters which are suitable particularly for subsequent integration in the illumination beam path 1 of already existing systems, preferably in a parallel beam portion. A geometrically structured filter comprising sector-shaped filter regions with different spectral filter characteristics whose sectors can have identical or different sector surface contents is also suitable but must be arranged in the vicinity of the aperture plane.

The blood vessels and their vessel-free environment are preferably identified by means of an image-processing algorithm at λᵢ=548 nm, and the intensities of their reflections in the images are determined and used as the basis for determining the oxygen saturation in the manner described in the following. This can be carried out based on individual image points, or an average is taken over a plurality of image points in a suitable manner.

The image points neighboring the blood vessels are used as an environment when no other vessel is detected therein. After the vessel direction is determined, an average is taken perpendicular to this direction over the reflection values of all of the image points associated with the blood vessel. In so doing, specular reflections on the blood vessel can be excluded from the averaging. It is also possible to determine in vessel direction a plurality of reflection values which are averaged perpendicular to the vessel direction and to use these in turn to form a (sliding) average. Averaging can also be carried out in the vessel environment in a similar manner.

A ratio of the optical densities ODR is used according to the invention. This ratio can be represented as a quotient of the logarithms of the ratios of the reflection Rᵤ of the vessel-free environment and the reflection R_{g} on a blood vessel at the measurement wavelength λₘ and at the reference wavelength λᵢ:

$$ODR = \frac{log \frac{{R}_{u} ( {λ}_{m} )}{{R}_{g} ( {λ}_{m} )}}{log \frac{{R}_{u} ( {λ}_{i} )}{{R}_{g} ( {λ}_{i} )}} \qquad ( 1 )$$

The oxygen saturation OS in the respective blood vessel in per cent is determined from (1) as a linear function:
OS=100−(ODR−a)/b−c+d, (2)
where the linear term a, as offset, and the slope b are to be determined empirically from readings over a sufficiently large quantity of blood vessels, for example, by comparing with normal values corresponding to a spectrometric method according to DE 199 20 157 A1. Variables c and d are correctives, where c serves to correct the dependency of the oxygen saturation on the vessel diameter and d serves to correct the dependency on the pigmentation of the local environment of the blood vessel.

The correctives c and d can be different for arteries and veins. Arteries and veins can preferably be distinguished based on a threshold for ODR which can accordingly be automated.

The correctives c and d are defined as linear functions of the vessel diameter g and pigmentation i from
c=(e−g)·f (3)
and
d=(h−i)·j (4)
where e and f, h and j are to be determined empirically as constants in corresponding series of measurements in such a way that the correlation between the vessel diameter and oxygen saturation vanishes.

Whereas the vessel diameter g can be measured separately, the melanin pigmentation of the fundus can be determined from the reflection values in the local environment of the blood vessel and is given by:

$$i = log \frac{{R}_{u} ( {λ}_{m} )}{{R}_{u} ( {λ}_{i} )} \qquad ( 5 )$$

A method according to DE 196 48 935 A1 is particularly suitable for determining the vessel diameter g. In this method, the vessel diameter g is determined based on vessel edge acquisition as the distance between photometric vessel edge centroids formed by interpolation with corrected oblique position of the vessel edges.

When the blood vessel is a vein, the following values result from empirically determined constants using illumination-side filtering with transmission ranges of λᵢ=548 nm±5nm and λₘ=610 nm±5 nm, and a color camera HVC 20A by Hatachi:
a=0.03556
b=0.0032
e=130
f=0.22
h=0.2339
j=55.5

On the other hand, for an artery the constants f and j take on the value of 0 so that the correctives c and d are omitted when determining the oxygen saturation. The values a and b are identical for veins and arteries.

In the method according to the invention, the classification of the blood vessels as veins and arteries is carried out automatically based on an ODR threshold value, where a vein is indicated when ODR>0.078 and otherwise an artery is indicated.

According to FIG. 3, in a method according to the invention, average values are determined for the intensity of the reflection on the artery or vein at the measurement wavelength of λₘ=610 nm and at the isosbestic wavelength of λᵢ=548 nm serving as reference wavelength after the blood vessels have been detected automatically through image-processing means or manually. Further, the intensity of the reflection is measured outside the blood vessels, i.e., in the vessel-free environment, and the average is formed from this. Edge zones with a wide variety of disrupting influences on the reflection relevant to oxygen saturation, e.g., influences of the vessel walls or shadows of the blood vessel on its substrate, are not taken into account when averaging. Specular reflections on the blood vessels can be identified and eliminated automatically by image-processing means or manually.

The method according to the invention makes it possible to show the vessel structure in the image of the biological object in which the oxygen saturation is coded, for example, in false colors. Vessel portions exhibiting a pathologically changed oxygen saturation can be determined by comparison with normal values and can be identified in the image. A statistical evaluation of the oxygen saturation of all blood vessels in the image in comparison with normal values allows a general diagnosis of existing pathologies.

Additional important diagnostic information is provided by the reaction of the oxygen saturation to physiological provocation or stimulation (e.g., by illuminating the eye with flicker light, inhalation of oxygen or carbogen by the patient).

For this purpose, the image-generating device according to FIG. 1 can have additional means which are also suitable for stimulation or provocation of the blood vessels such as a controllable optical light manipulator 12 which is arranged in the common illumination beam path 1 next to the filter device 3 and whose control module 13 has an interface to the controlling and evaluating computer 8 (shown in dashes).

The light manipulator 12 which is controllable in a variety of ways by programming is shared between all of the illumination sources and, by modifying primary light, in this case the continuously emitting illumination source 2 and the strobe illumination source 10, generates secondary light.

The light manipulator is suitable for programmable modification of the light of at least one light source with respect to its intensity curve and/or time curve in a temporally defined relationship with the adjustments of the at least one light source, the image recording and the evaluation for adaptively accommodating to the examination task. The secondary light can be used for illumination and for selective provocation or stimulation.

Therefore, multifunctionality can be achieved by influencing the illumination by means of an individual element arranged in the illumination beam path in that the light characteristics of the light guided in the illumination beam path are changed so as to be adapted to function.

By recording and evaluating pulse-synchronized sequences of images, systolic and diastolic differences in oxygen saturation can be obtained as diagnostic features. By combining the measured oxygen saturation with other local or general characteristic values of microcirculation such as vessel diameter, blood flow rate or blood pressure, the oxygen supply and metabolism in the tissue can be described in detail.

While the foregoing description and drawings represent the present invention, it will be obvious to those skilled in the art that various changes may be made therein without departing from the true spirit and scope of the present invention.

What is claimed is:

1-24. (canceled)

25. A method for the spectral photometric determination of the oxygen saturation of the blood in optically accessible blood vessels comprising:
determining the intensity of the reflection of the blood vessels and their vessel-free environment based on at least two spectrally different images and on an empirically determined relationship between the oxygen saturation and a ratio of the intensities of the reflection of the blood vessels and their vessel-free environment; and
said determining step further comprising the steps of illuminating the blood vessels and their environment simultaneously by at least one measurement wavelength and at least one reference wavelength of an illumination beam for recording the spectrally different images, and tuning every measurement wavelength and reference wavelength, respectively, to a color channel of a color camera used to record the images in order to be received by this color channel.

26. The method according to claim 25, wherein the measurement wavelength is a wavelength at which the reflection of oxygenated and reduced hemoglobin differs, and the reference wavelength is an isosbestic wavelength of the hemoglobin.

27. The method according to claim 26, wherein the oxygen saturation is determined as a linear function of the quotient of the logarithmized reflection ratios in the vessel-free environment and on the blood vessel at the measurement wavelength and at the isosbestic wavelength, and wherein the slope and linear term of the linear function are determined empirically from readings at a plurality of blood vessels.

28. The method according to claim 27, wherein disturbances caused by a dependency of the oxygen saturation on the vessel diameter and on the pigmentation of the environment of the blood vessels are compensated by correctives that are empirically determined and taken into consideration additively.

29. The method according to claim 28, wherein the corrective for compensating for the influence of the vessel diameter is a linear function of the vessel diameter, and its slope and linear term are determined empirically.

30. The method according to claim 28, wherein the corrective for compensating for the influence of the pigmentation of the environment of the blood vessels is a linear function of the pigmentation, and its slope and linear term are empirically determined.

31. The method according to claim 30, wherein the pigmentation of the environment of the blood vessels is determined by the logarithm of the quotient of the reflection values of the environment of the blood vessels at the measurement wavelength and at the isosbestic wavelength.

32. The method according to claim 25, wherein arteries and veins are distinguished based on the quotient of the logarithmized reflection ratios in the vessel-free environment of the blood vessel and on the blood vessel at the measurement wavelength and at the isosbestic wavelength.

33. The method according to claim 25, wherein the blood vessels, their direction and their vessel-free environment are detected automatically by image-processing means or manually.

34. The method according to claim 33, wherein, perpendicular to the direction of the blood vessel, an average is taken over the reflection values of all of the image points associated with the blood vessel.

35. The method according to claim 34, wherein a plurality of reflection values which are averaged perpendicular to the direction of the blood vessel is determined along the direction of the blood vessel, and the average is taken over these averaged reflection values.

36. The method according to claim 35, wherein specular reflections on the blood vessels are identified and eliminated automatically through image-processing means or manually.

37. The method according to claim 25, wherein the oxygen saturation is determined in reaction to physiological provocation or stimulation.

38. The method according to claim 37, wherein the physiological provocation or stimulation is brought about by flicker light.

39. The method according to claim 38, wherein light from at least one light source is modified through programming techniques by a light manipulator arranged in an illumination beam path of an image-generating device, and wherein the modified light is used for illumination and for selective provocation or stimulation.

40. The method according to claim 37, wherein the physiological provocation or stimulation is brought about by inhalation of oxygen by the test subject.

41. The method according to claim 37, wherein the physiological provocation or stimulation is brought about by inhalation of carbogen by the test subject.

42. The method according to claim 25, wherein an image is prepared of the structure of the blood vessel in which the oxygen saturation is coded.

43. The method according to claim 25, wherein an image is prepared of the structure of the blood vessel in which the blood vessels with pathological oxygen saturation are marked.

44. The method according to claim 25, wherein a plurality of oxygen saturation values are determined from a tissue area, and results are obtained therefrom by statistical evaluation for oxygen supply and for oxygen consumption in the tissue area.

45. The method according to claim 25, wherein systolic and diastolic differences in oxygen saturation are obtained as diagnostic features by recording pulse-synchronized sequences of images.

46. The method according to claim 25, wherein the oxygen saturation is used in combination with other local or general characteristic values of microcirculation, such as vessel diameter, blood flow rate or blood pressure, to determine the oxygen supply and metabolism in a tissue region.

47. A method for the spectral photometric determination of the oxygen saturation of the blood in optically accessible blood vessels comprising:
determining the intensity of the reflection of the blood vessels and their vessel-free environment based on at least two spectrally different images and on an empirically determined relationship between the oxygen saturation and a ratio of the intensities of the reflection of the blood vessels and their vessel-free environment; and
said determining step further comprising the steps of determining the oxygen saturation as a linear function of the quotient of the logarithmized reflection ratios in the vessel-free environment and on the blood vessel at a measurement wavelength at which the reflection of oxygenated and reduced hemoglobin differs and at an isosbestic wavelength of the hemoglobin as reference wavelength, and determining the slope and the linear term of the linear function are determined empirically from readings at a plurality of blood vessels.

48. The method according to claim 47, wherein disturbances due to a dependency of the oxygen saturation on the vessel diameter and on the pigmentation of the environment of the blood vessels are compensated by empirically determined correctives which are to be taken into account additively.
